(19) United States
(12) Patent Application Publication
(10) Pub. No.: US 2025/0179560 A1
(43) Pub. Date: Jun. 5, 2025

(54) METHOD FOR DETECTING NUCLEIC ACID AMPLIFICATION PRODUCTS USING BLOCKING AGENTS

(71) Applicant: Rarity Bioscience AB, Uppsala (SE)

(72) Inventors: Ulf LANDEGREN, Uppsala (SE); Lei CHEN, Storvreta (SE)

(73) Assignee: Rarity Bioscience AB, Uppsala (SE)

(21) Appl. No.: 18/844,573

(22) PCT Filed: Mar. 8, 2023
(86) PCT No.: PCT/EP2023/055898

§ 371 Date: Sep. 6, 2024

(30) Foreign Application Priority Data
Mar. 8, 2022 (GB) 2203185.0

(51) Int. Cl. C12Q 1/682; C12Q 1/6832; C12Q 1/6886

(52) U.S. Cl. CPC C12Q 1/682; C12Q 1/6832; C12Q 1/6886; C12Q 2600/156

(57) ABSTRACT

The present invention provides a method for detecting two or more differently labelled high molecular weight nucleic acid products in a mixture, wherein said products are detected by detecting one or more labels which are attached directly or indirectly to each product, said method comprising including in the mixture for detection a resolving agent to facilitate resolution of the differently labelled products, wherein said resolving agent acts to inhibit interaction between different labels.

### FIELD

The present disclosure and invention concerns the field of nucleic acid detection. Particularly, the present disclosure and invention relates to a method for detecting the products of rolling circle amplification (RCA) or other amplification or hybridisation reactions which produce large nucleic acid products. According to the method herein, the resolution of differentially labelled RCA or other nucleic acid amplification products present in the same reaction mixture may be improved. This has particular applicability in the detection of target nucleic acid sequences.

### BACKGROUND

The detection of target nucleic acid sequences has applications in many different fields, including notably clinically, for personalised medicine and in the diagnosis, prognosis and/or treatment of disease, such as cancer, infectious diseases and inherited or genetic disorders, as well as in research and biosecurity.

The target nucleic acid sequence may readily be detected using labelled hybridisation probes, but simple hybridisation probes have relatively high lower detection limits, and cannot readily be used to discriminate between similar nucleic acid sequences. To increase sensitivity, target nucleic acid molecules containing target sequences may typically be amplified, to increase the amount of target sequence available for detection. Any of a variety of techniques known in the art may be used for the amplification, but particularly rolling circle amplification (RCA) has many advantages.

RCA is an isothermal amplification technique which utilises a strand displacement polymerase enzyme and requires a circular amplification template. Amplification of the circular template provides a concatenated RCA product, comprising multiple copies of a sequence complementary to that of the amplification template. Such a concatemer typically forms a ball or “blob”, which may readily be visualised and detected, and thus RCA-based assays have been adopted for the detection of nucleic acids, and indeed, more generally, as reporter systems for the detection of any target analyte. Both target nucleic acids, which may themselves be circularised directly, or probes, or reporter nucleic acids more generally may provide template nucleic acid circles for RCA.

RCA-based assays have been described which rely on secondary amplification of the initial RCA product, to increase the amount of product which is detected, and thereby to provide amplification of the signal in the assay. These include, for example, hyberbranched RCA which generates many unclustered subsequent RCA products through the strand displacement activity. More recently, so-called “SuperRCA” (sRCA) reactions have been developed which comprise 2 or more rounds of RCA amplification, wherein the product of the second RCA reaction, the second RCP, is linked to that of the first, namely to the first RCP. One version of such a sRCA method is described in WO2014/0796209. In this method, a probe capable of providing or functioning as a primer is hybridised to an initial RCA product and is used to prime the amplification of a second RCA template circle which hybridises to the “primer-probe”. The second RCA template may be generated by circularisation of a padlock probe which hybridises to the “primer-probe”.

In WO 2015/071445, an alternative sRCA method, termed “Padlock sRCA”, is described, in which a padlock probe is used to bind directly to the initial RCA product (RCP). Thus, a padlock probe may be hybridised to each monomer repeat of the first RCP (i.e. to each complimentary copy of the first RCA template used to generate the first RCP), or at least to a high proportion thereof. The hybridised padlock probes may then be circularised by intramolecular ligation and serve as second RCA templates to generate a second RCP which is linked to the first.

The sensitivity afforded by such RCA, and particularly sRCA, methods means that they have found application in the detection of rare, or low-abundance, targets, most notably in the case of detection of mutations which may be rare events, or where limited amounts of sample are available. It is frequently convenient or desirable to perform such detection assays in multiplex, where multiple target nucleic acid sequences present in one sample, or one reaction mixture, are detected together. This requires the individual RCPs generated for each separate target to be detected in a mixture in a manner in which they may be distinguished from one another.

RCPs are typically labelled for detection. This may involve the incorporation of labels into the product, for example, by incorporating labelled nucleotides during the synthesis of the RCP, or by the use of intercalating dyes to label the RCPs. A common way of labelling a RCP is by the use of labelled detection oligonucleotides which hybridise to the RCP. Thus, an RCP may be labelled directly or indirectly. An RCP, being a long concatemer comprising many, typically hundreds and in some cases thousands, of repeats of complementary copies of the RCA template circle, provides a similarly large number of sites where a label may be incorporated or attached. A sRCA product comprises very many such concatemers, since a padlock probe may be hybridised (directly or indirectly) to a high proportion of the monomer repeats of the first RCP, and each such padlock probe may lead to the generation of a second RCP. The resulting sRCA product (sRCP) may thus be labelled with very many labelled molecules.

A problem common to any multiplex detection techniques which rely on the use of labels to distinguish large nucleic acid products is the interference which may occur between the labels in or on the different products. In particular, it has been found that the labels attached to individual RCPs present in the same mixture may stick to one another, impeding resolution of the individual RCPs. Since a particular advantage of RCA-based detection techniques is that individual RCPs may individually be detected and quantified, this presents a problem that may confound the assay, and reduce the level of multiplexing that may be performed.

This problem is not limited to the detection of RCPs and applies to any large, or high molecular weight, nucleic acid product which comprises or provides a high number of labelling sites. This may include the product of amplification methods such as hybridisation chain reaction (HCR), where large numbers of monomers, each comprising a label or a detectable sequence to which a detection oligonucleotide may be hybridised, are incorporated. It is known, for example, that signal amplification methods may involve the build-up of a sequence of probes which hybridise to each other, to provide multiple binding sites for labelled detection probes, or other labelling systems, as exemplified by the RNAscope™ technology, as described in WO2011/094669 for example. Whilst RNAscope™ was developed for in situ hybridisation for detection of RNA, it exemplifies the principle of using sandwich-type, or intermediate, hybridisation probes each providing multiple binding sites for labelled detection probes, to generate a detectable nucleic acid product comprising multiple labels. Such nucleic acid products may be viewed as so-called “nucleic acid accumulations” (NAAs), which comprise multiple target sites for detection or, in other words, multiple detectable sites.

### SUMMARY

The present inventors have found that the resolution of differentially-labelled nucleic acid products such as RCPs and other NAAs may be improved by the use of resolving agents, which when included together with the labelled nucleic acid products, reduce the interference between labels on different products.

Such resolving agents are typically proteins, particularly inert proteins, such as are conventionally used as blocking agents in assay methods to prevent or reduce non-specific binding of assay reagents. However, they may also include non-proteinaceous agents such as detergents or synthetic polymers. Whilst the use of such blocking agents is widely known, they have not previously been described for use in the steps of labelling or detecting the products of assay reactions. The presently proposed methods represent a new use for such blocking agents.

Accordingly, in a first aspect, provided herein is a method for detecting two or more differently labelled high molecular weight nucleic acid products in a mixture, wherein said products are detected by detecting one or more labels which are attached directly or indirectly to each product, said method comprising including in the mixture for detection a resolving agent to facilitate resolution of the differently labelled products, wherein said resolving agent acts to inhibit interaction between different labels.

Particularly, the interaction between labels on different nucleic acid products is inhibited.

Particularly, the labels are attached at a high copy number.

The nucleic acid products to be detected are thus high molecular weight nucleic acid products which comprise multiple labels per product. In particular, the nucleic acid product comprises multiple sites for incorporation or attachment of a label. The labelled nucleic acid product carries, or comprises, multiple copies of the label(s).

Rather than referring to them as “high molecular weight” nucleic acid products, the nucleic acid products to be detected may alternatively be referred to as nucleic acid accumulations (NAAs), accumulated nucleic acid products or nucleic acid clusters. In particular, the nucleic acid products to be detected may be defined as NAAs, accumulated nucleic acid products or nucleic acid clusters which each comprise multiple copies of labels which distinguish them from one another.

Different nucleic acid products are differently labelled, such that they can be distinguished or discriminated from one another. In an embodiment, each nucleic acid product to be detected may be labelled with one label (in the sense of one species of label), and different products have different labels. However, to increase multiplexing capacity, and to allow a greater number of different products to be distinguished from each other in the mixture, two or more labels (in the sense of species of label) may be applied to each product in different combinations and/or ratios, such that each product has a different combination and/or a different ratio of labels.

In an embodiment, the nucleic acid products are nucleic acid amplification, nucleic acid polymerisation or nucleic acid hybridisation products. In particular, they may be products which comprise multiple copies, or repeats of a nucleic acid monomer unit or of an oligonucleotide molecule.

In an embodiment, the nucleic acid products are RCA products (RCPs) or hybridisation chain reaction (HCR) products, or hybridisation products which result from the hybridisation of multiple hybridisation probes to proximal locations on the same nucleic acid molecule.

Thus, in one embodiment, the nucleic acid products are composed of multiple hybridisation probes hybridised together to form one hybridised nucleic acid product. Accordingly, the nucleic acid product may represent an accumulation of multiple hybridisation probes. In particular, the nucleic acid product may comprise a scaffold nucleic acid to which are hybridised multiple hybridisation probes.

The nucleic acid products may be generated as detectable signals, or markers, for the detection of target analytes of a detection method (i.e. for the target analytes of an assay). In other words, the nucleic acid products may be generated during a method for detecting a target analyte. They may be viewed as the reaction products of a detection assay. In other words, they may represent the signal products, or detectable products, of an assay.

More particularly, the nucleic acid product may be generated in a method for detecting a target nucleic acid sequence in a target nucleic acid molecule. The target nucleic acid sequence, or the target nucleic acid molecule, may be a target analyte of the detection method, or a reporter nucleic acid for an analyte of the detection method. Thus, the nucleic acid product may be generated in an assay method for the detection of any analyte, nucleic acid or non-nucleic acid, e.g. a protein analyte.

In an embodiment, the nucleic acid products are labelled by hybridised detection oligonucleotides which each comprise a detectable label.

In an embodiment, the monomers, e.g. HCR monomers or hybridisation probes, which make up the nucleic acid product comprise a label.

In an embodiment, the label is spectrophotometrically detectable. In an embodiment, the label is a fluorescent, coloured, colorimetric, particulate or metallic label.

In an embodiment, the nucleic acid products are detected by microscopy or by flow cytometry.

In an embodiment, the resolving agent is added to the mixture of nucleic acid products.

In an embodiment, the resolving agent is present in the reaction mixture when the nucleic acid products are labelled.

In an embodiment, the resolving agent is present in the reaction mixture when the nucleic acid products are generated, and remains in the labelled mixture which is detected.

In a further aspect, provided herein is a reaction mixture comprising (i) two or more differently labelled high molecular weight nucleic acid products, wherein said products are labelled by multiple copies of one or more labels which are attached directly or indirectly to each product, and (ii) a resolving agent which facilitates resolution of the differently labelled products, wherein said resolving agent acts to inhibit interaction between different labels.

In a still further aspect, provided herein is a method for detecting two or more target nucleic acid sequences in one or more nucleic acid molecules in a sample, said method comprising:
(a) for each target nucleic acid sequence, generating a high molecular weight nucleic acid product as a detection assay reaction product for that sequence, and creating a reaction mixture comprising the resulting nucleic acid products for the two or more target nucleic acid sequences;
(b) providing each nucleic acid product of (a) with multiple copies of one or more labels, wherein each nucleic product is differently labelled and can be distinguished from another nucleic acid product, and wherein the labels are provided during the generation of the nucleic acid products in (a) or after, thereby generating a detection mixture comprising two or more differently labelled nucleic acid products, wherein a resolving agent is included in the detection mixture to facilitate resolution of the differently labelled products, wherein said resolving agent acts to inhibit interaction between different labels;
(c) detecting the labelled nucleic acid products in the detection reaction mixture, thereby detecting the target nucleic acid sequences.

In an embodiment, in step (b), the labels are provided after the nucleic acid products have been generated. Accordingly, in such an embodiment, step (b) is a separate labelling step performed after step (a). In such an embodiment, step (b) may comprise contacting the reaction mixture from (a) with a labelling mixture which comprises the resolving agent. Alternatively or additionally, the resolving agent may be included in the reaction mixture(s) for generating the nucleic acid products (i.e. in step (a)).

In an embodiment, the labelling mixture comprises labelled detection oligonucleotides which are capable of hybridising to the nucleic acid products. It will be understood in this regard that different detection oligonucleotides are provided for each nucleic acid product, wherein different detection oligonucleotides hybridise to different nucleic acid products. In other words, each nucleic acid product has a cognate detection oligonucleotide which is capably of hybridising specifically to it.

The nucleic acid products may be any of the nucleic acid products above. In an embodiment, the nucleic acid reaction product is a RCP.

In an embodiment of any of the aspects above, the RCP is generated by RCA of a circularised padlock probe which is specific for the target nucleic acid sequence. In another embodiment, the RCP is generated by RCA of a circularised target nucleic acid sequence or an amplicon thereof, e.g. a PCR amplicon thereof. In any of these embodiments, the RCA may comprise two or more rounds of an RCA reaction. In other words, the RCA reaction may be a sRCA reaction. Accordingly, the nucleic acid product may be a second or further generation RCP, or a sRCA product.

### DETAILED DESCRIPTION

The methods herein address the problem of interference between labels in or on different nucleic acid products generated to detect different nucleic acid targets in detection assays.

As noted above, detection assays are frequently performed in multiplex to detect multiple analytes, or targets, in a single sample or mixture. This requires the signals generated for the different targets to be distinguished from one another. This may typically be achieved by using different labels for different targets, or different combinations or ratios thereof, such that each target has a particular signal associated with it.

As further noted above, many detection assays rely on signal amplification to improve the sensitivity and accuracy of the assay, and large nucleic acid products which may readily be labelled and detected, such as RCPs, are generated as the ultimate reaction products which are detected in many assay methods as a means of detecting the target analyte of the assay. In other words, the reaction product which is generated as the signal, or marker, of the assay is typically a large nucleic acid product, which can comprise, or be labelled with, a high copy number of labels.

Such multiplex detection methods are typically used to detect variants of target nucleic acid sequences. Target nucleic acid sequences may commonly occur in variant forms, for example allelic variants, or mutant and wild-type sequences, and it may be desirable to detect which variant is present. Thus, the target nucleic acid sequence may be one of a number of different variants of the nucleic acid sequence which may occur in a target nucleic acid molecule. It is important to be able to distinguish different variants from one another, and thus for the signals generated for each variant to be able to be detected distinctly. For example, a sample may contain both mutant and wild-type sequences and it may be important to detect and quantify these separately, and this is also important to be able to determine allele frequencies correctly.

In the course of developing the method herein, it was found that double positives, with signals for both mutant and wild-type sequences, were frequently observed. These arose as a result of labels (e.g. fluorophores) on the “mutant” nucleic acid product, sticking to or otherwise interfering with labels on the “wild-type” product present in the same reaction mixture. By including a blocking agent that is typically used to prevent non-specific binding of proteins to surfaces in the reaction mixture for detection, such interference could be reduced or prevented. Thus, the accuracy of detection, and the sensitivity and specificity of the detection method may be improved.

The nucleic acid products which are generated for detection as the signals or markers for the detection targets, or assay analytes, are defined above as high molecular weight nucleic acid products, or alternatively as nucleic acid accumulations (NAAs) or accumulated nucleic acids. They are in effect nucleic acid clusters, which may be composed of a single nucleic acid molecule, or of multiple separate nucleic acid molecules, or oligonucleotides, which are hybridised together.

By “high molecular weight” it is meant a large nucleic acid product. Typically, this may include products in the μm size range (e.g. at least 0.5 μm), which may readily be visualised by microscopy, or detected as a particle, for example, by flow cytometry. Thus, the nucleic acid product may alternatively be defined as being of a size which is detectable per se by microscopy or flow cytometry. Generally speaking, “high molecular weight” may be taken to indicate at least 500 kDa, e.g. at least 1 megadalton, or at least 10, 50, 100, 200, or 500 megadaltons, or even at least 1 gigadalton. Alternatively, this may be defined as a nucleic acid product which comprises at least 1, 5, 10, 20, 50, or 100 megabases. Alternatively, a high molecular weight nucleic acid product may be defined as being capable of carrying at least 15,000, 20,000 or 25,000 copies of a label, or labelling reagent, for example, including even in the range of 100,000 to 1 million copies.

In an embodiment, where the nucleic acid product comprises or is made up of monomer units (e.g. monomer repeats such as in a RCP, or monomers which are joined such as in a HCR product etc.), this may be at least 100, 200, 300, 400 or 500 monomers. For a sRCA product comprising at least 2 generations of RCP, this may be at least 100×100 monomers, and so on, up to at least 500×500 monomers. However, with extended RCA reaction times (e.g. up to 1 hr) it is not precluded that even larger products may be obtained, for example, with up to 700, 800, 900, or 1,000 monomers per RCA cycle.

In an embodiment, the term “high molecular weight nucleic acid product”, or NAA, does not typically include a PCR product. In particular, it does not include a PCR product comprising a single repeat, or copy, of a target sequence to be detected.

Since they are large, the nucleic acid products may accommodate multiple copies of the labels which are used to label them. In other words, they comprise multiple sites for incorporation or attachment of a label, or a moiety carrying a label (a labelled moiety). In an embodiment, such a site may be a sequence, termed a detection sequence herein, which provides a binding site for a detection probe (i.e. a detection sequence as referred to herein). The detection sequence can be viewed as a tag sequence. The detection probe may comprise a label. It will be seen that in the case of a nucleic acid product which comprises multiple monomers, each monomer may comprise a detection sequence, and thus a binding site for a label (specifically for a labelled detection oligonucleotide). Similarly, a nucleic acid product may comprise multiple hybridisation probes which each comprise a detection sequence.

The term “multiple” or “multiplicity” as used herein means two or more, for example, 3, 4, 5, 6, 8, 9, 10, 15, 20, 25, 30, 40, 50, 60, 70, 80, 90, or 100 or more. Indeed, in the case of many types of nucleic acid product, this may comprise a thousand or more detection sequences and hence binding sites for labels, or labelled detection oligonucleotides more precisely. Thus, “multiple” in the context of label copy number, or binding/attachment sites for labels, or detection sequences etc. may include at least 200, 300, 400, 500, 600, 700, 800, 900 or 1000, or multiples of these, as set out above for the possible number of monomers, e.g. in a sRCA product comprising first and second RCPs.

The nucleic acid product (or NAA) is characterized by comprising a detection sequence, or more generally a labelling site, at high concentration or high copy number at a spatially defined site or position. As indicated above, such a product may originate from localized amplification or polymerization reactions (such as RCA or HCR), or from the hybridisation of multiple hybridisation probes at proximal locations on the same nucleic acid molecule. Such a hybridisation system may allow a branched nucleic acid structure to be built up, for example, akin to a RNAscope™ product as mentioned above. Thus, a template or scaffold molecule may be provided which hybridises to the target nucleic acid sequence, and which comprises multiple binding sites for further hybridisation probes. These may comprise detection sequences, or they may comprise binding sites for further hybridisation probes etc. Thus, a “layered” or “branched” structure may be made up, composed of multiple hybridisation probes. The hybridisation probes, or a subset thereof (e.g. the last or final hybridisation probes added to the structure), may comprise a detection sequence. The nucleic acid product may in such an embodiment be referred to as a hybridisation assembly.

Thus, the nucleic acid products may be nucleic acid amplification, nucleic acid polymerisation or nucleic acid hybridisation products.

In certain embodiments, they may be RCA or HCR products (RCPs or HCRPs), but they may be produced by any amplification reaction which can be employed or adapted to produce such a product, including for example, PCR, recombinase polymerase amplification (RPA), helicase-dependent amplification (HAD), LAMP, multiple strand displacement amplification (MDA) etc. Amplification methods such as MDA may be used to generate branched amplification products.

Whether produced by an amplification, polymerisation, or hybridisation reaction etc., the nucleic acid products can be seen as comprising multiple monomers, joined together, whether covalently or by hybridisation, wherein the monomers may each comprise a detection sequence which can be labelled, for example by hybridisation of a labelled detection probe (also referred to as a “detection oligonucleotide” herein). The monomer may be a repeat in a RCP or other amplification product, a HCR monomer in a HCRP, or a hybridisation probe in a hybridisation product.

The nucleic acid product may have a linear or a branched structure. In the case of a linear product such as an RCP, as noted above, this may be coiled into a ball, or blob, a structure which is a discrete visualisable entity.

The nucleic acid product is typically a DNA product. However, it may also be composed of or may comprise other nucleic acids, natural or synthetic. Thus, it may, for example, be a chimeric construct comprising both RNA and DNA. The nucleic acid product may be made up of ribonucleotides and/or deoxyribonucleotides as well as synthetic nucleotides that are capable of participating in Watson-Crick type or analogous base pair interactions. Thus, the nucleic acid product may be or may comprise, e.g. LNA, PNA or any other derivative containing a non-nucleotide backbone.

The resolving agent may be any of the blocking reagents typically used to block non-specific binding of assay reagents in detection methods (e.g. in immunoassays), for example, to coat or block non-specific binding sites on the surfaces of reaction vessels etc. Such a blocking agent is typically a protein, but non-proteinaceous blocking agents are also known.

Thus, the resolving agent may be proteinaceous, that is, it may be or may comprise a protein. Conveniently, this will be an inert protein. By “inert” it is meant that the protein does not interfere or take part in any of the reactions performed in the detection method, or in the detection mechanism more generally. It may nonetheless inhibit (i.e. reduce or prevent) any interaction which may occur between label molecules or moieties. In particular, it may inhibit labels from binding to, or associating with, one another.

The resolving agent may be a protein with very low, negligible, or no specific binding affinity for the target analyte of the detection method, i.e. it is not capable of binding specifically to the target analyte. It may be a single species or type of protein, or a mixture of different proteins, e.g. different protein types or different species of the same type (the term “species” as used in this context does not have a taxonomical meaning, but rather is intended to denote a protein of a particular specific type).

The resolving agent may generally be a stable and abundant or common protein such as a serum protein, a milk protein, or gelatin. Thus, for example, it may be provided as casein, or as dry milk powder, e.g. non-fat dry milk, which is commonly used in blocking applications. A serum protein may be a single specific type of serum protein or may comprise a plurality of serum proteins of different types and structures. It may be or comprise a serum albumin and/or a globulin, and may be provided as an individual protein, a protein mixture or as serum.

Serum protein as a generic component comprises both globulin and albumin proteins and is derived from blood plasma. Serum may be defined as the fraction of blood that remains when the cells and platelets have been removed. More specifically, blood serum may be defined as the fraction of blood plasma that does not contain cells, fibrinogen or any other blood clotting factors. Thus, the serum protein may be any protein which may be obtained (or obtainable) from serum or which may occur in serum. It may be a single protein which may occur in or be obtained from serum, or a mixture of such proteins, or it may be a protein fraction or protein component from serum. The resolving agent may comprise serum protein in general, that is, it may be represented by the serum protein component of serum generally, without separation of a particular protein component. In other words, it may be a mixture of the proteins which occur in serum, and which may be separated therefrom. Thus, the resolving agent may comprise both globulin and albumin, and preferably γ-globulin (immunoglobulin) and/or serum albumin. Said serum protein may be from a single blood source or multiple blood sources, i.e. from different animal individuals and/or from different types of animal. Thus, serum proteins of or from different species may be used, e.g. from any mammalian species. Although natural sources of serum protein are convenient, a serum protein may be synthetically derived or obtained, for example, by recombinant expression, or by derivatisation of a naturally occurring protein. Thus, included within the term “serum protein” are not only any protein which may occur in the serum naturally, but also variants and derivatives thereof, for example, fragments or truncated proteins, chemically-modified proteins or polypeptides, or variants obtained by genetic engineering, for example, polypeptides based on the amino acid sequence of a naturally-occurring serum protein, but comprising one of more amino acid substitutions, additions and/or deletions etc. Also included are equivalent or corresponding proteins, which may not necessarily occur in serum but which are structurally or functionally equivalent.

Globulin proteins, pseudoglobulins and euglobulins, are found widely throughout the animal and plant kingdoms and are characterised by their physical properties such as solubility and electrophoretic migration, e.g. pseudoglobulins are soluble in both water and dilute salt solutions, whilst euglobulins are insoluble in water and soluble in dilute salt solutions. Both sub-classes of globulin are coagulable by heat. Any such globulin protein may be used as the resolving agent. Thus, generally speaking, the resolving agent may comprise any globulin protein. As noted above albumin proteins may also be used, and accordingly the resolving agent may be or may comprise any albumin protein.

Prominent sources of globulin proteins are blood plasma and serum, milk, muscle and plant seeds. In particular, the term “globulin” encompasses a heterogeneous group of proteins found in blood serum, which are classified as having a high molecular weight, and both solubility and electrophoretic migration rates lower than for albumin.

Thus, in one embodiment, the resolving agent is blood serum globulin, which may comprise four main classes of protein namely: α-1 globulins, α-2 globulins, β-globulins and γ-globulins. However, it will be understood by a person of skill in the art that where serum has been treated to remove fibrinogen and other clotting factors, serum protein comprises only a subset of globulins, predominantly the γ-globulins. A resolving agent may therefore comprise a serum protein preparation where the globulin fraction of the serum protein comprises at least 70% γ-globulins, e.g. at least 80% or at least 90% γ-globulins.

The α-globulins are characterised by their ability to be highly mobile in alkaline or electrically charged solutions and include α-1 antitrypsin and serum amyloid A (α-1 globulins) and haptoglobin and ceruloplasmin (α-2 globulins). β-globulins are characterised by being less mobile in alkaline or electrically charged solutions than α-globulins, but more so than γ-globulins and include plasminogen and transferrin. Thus, γ-globulins are less mobile in alkaline or electrically charged solutions than both the α- and β-globulins and include as the predominant type of protein, the immunoglobulins (antibodies). Antibodies are well described in the art and may be classified in various groups, commonly IgG, IgE, IgD (all monomers), IgA (dimers) and IgM (pentamers).

In a particular embodiment, the resolving agent comprises γ-globulin and particularly immunoglobulin, and more particularly IgG. In a convenient embodiment, the resolving agent is bulk IgG, i.e. immunoglobulin purified from blood serum comprising a plurality of immunoglobulin (IgG) proteins having a range of binding specificities and affinities. Bulk IgG thus comprises different IgG proteins having different specificities, i.e. a mixture of IgG proteins with different specificities. Whilst it is contemplated that the resolving agent may comprise an immunoglobulin protein with a specific structure, i.e. binding characteristics, this feature is only practical if the binding properties of that immunoglobulin do not interfere with the detection of the target analyte, e.g. if the target analyte of the detection method is a protein, and if the analyte remains in the reaction mixture which is subjected to the labelling and detection steps. The important point is that any binding of the blocking reagent to the target analyte is not such as to interfere with the performance of the assay. Thus, it may, depending on the assay format, be advantageous if the immunoglobulin component e.g. the IgG or bulk IgG, does not have or has low (or very low, or negligible or undetectable) binding activity towards the target analyte.

In an alternative embodiment, the resolving agent is or comprises a serum albumin protein. Serum albumin may be derived from a single source, e.g. bovine serum albumin (BSA), human serum albumin (HSA), porcine serum albumin (PSA), etc. or may be a combination of different serum albumin proteins.

In other embodiments, the resolving agent may be a DNA binding protein, e.g. single-stranded binding protein (SSB).

Alternatively, the resolving agent may be non-proteinaceous. In such embodiments, it may be or may comprise a detergent, e.g. a neutral detergent, such as CHAPS, Triton X-100 or Tween-20. It may also be a non-protein polymer, for example a polyvinyl alcohol, e.g. polyvinyl pyrrolidone (PVP).

Although a major benefit of the resolving agent is in reducing interference between labels during the detection step, there may also be a benefit from including the resolving agent during earlier steps, including notably the step of generating the high molecular weight nucleic acid product. Thus, the presence of the resolving agent in the reaction solution may have utility throughout the whole assay reaction which leads to generation of the nucleic acid product for detection. For example, the resolving agent may also be beneficial in preventing the nucleic acid products (e.g. RCA products) from colliding with each other, to optimize the rate of the generation (e.g. RCA) reaction and reduce errors in the process. Accordingly, the resolving agent may be added before the labelling and detection steps, for example, before or during the step of generating the product (e.g. the amplification/polymerisation/hybridisation reaction which generates the product, such as the RCA reaction).

However, when the resolving agent is present in high concentrations during certain reactions, such as, for example, in the RCA reaction mixture, the resolving agent may reduce the rate of, or inhibit, the generation (e.g. RCA) reaction. Accordingly, in preferred embodiments, the concentration of the resolving agent is at a level which does not affect, or interfere with, the nucleic acid product generation (e.g. RCA) reaction. For example, the maximum concentration of a resolving agent during the step of generating the nucleic acid product (e.g. the RCA reaction) may be up to 0.05, 0.06, 0.07, 0.08, 0.09 or 0.1% (by weight, or w/v). In one embodiment, the maximum concentration of the resolving agent is 0.1% (by weight, or w/v). Alternatively, in another embodiment, the resolving agent is present at a concentration of less than 0.1% (by weight, or w/v). In another embodiment, the resolving agent is present at a concentration of up to (or no more than) 0.09 or 0.08 or 0.075% (by weight, or w/v). This is particularly the case where the resolving agent is proteinaceous.

In contrast, the concentration of the resolving agent does not need to be limited during the labelling or detection steps, and therefore the concentration of the resolving agent may, for example, be higher in the labelling or detection steps than during the reaction steps which generate the nucleic acid product, for example, if additional resolving agent were to be added to the mixture during the labelling or detection steps.

During the detection step, the concentration of resolving agent should be sufficiently high to allow improved resolution of detection of the nucleic acid products.

In an embodiment, particularly in the case of a proteinaceous resolving agent, the minimum effective concentration of a resolving agent during the detecting step may be, for example, at least 0.001, 0.002, 0.003, 0.004, or 0.005% (by weight, or w/v). In one embodiment, the minimum concentration of the proteinaceous resolving agent is 0.001% (by weight, or w/v).

It may be advantageous for ease of use to maintain similar concentrations of resolving agent throughout the entire assay, or detection method, process.

Accordingly, the concentration of the proteinaceous resolving agent in the reaction mixture may range from 0.001-0.1%, or more particularly, 0.001-0.09% or 0.001-0.08% e.g. 0.002-0.09%, 0.002-0.08%, 0.002-0.07%, 0.002-0.06%, 0.002-0.05%, 0.003-0.08%, e.g. 0.004-0.07% (by weight, or w/v). Indeed, the range may be between any of the integers mentioned above, for example, between any of the minimum and maximum values stated above. In a particular embodiment, the concentration of the proteinaceous resolving agent may, for example, range from 0.004-0.02% (by weight, or w/v). In a preferred embodiment, the concentration of the proteinaceous resolving agent may be 0.01-0.02%, e.g. 0.01 or 0.02% (by weight, or w/v).

In one embodiment, serum albumin protein may be used as the resolving agent. For example, the maximum concentration of serum albumin protein during the reaction which generates the nucleic acid product (e.g. during the RCA reaction) may be up to 0.05, 0.06, 0.07, 0.075, 0.08, 0.09 or 0.1% (by weight, or w/v). In one embodiment, the maximum concentration of serum albumin protein is 0.1% (by weight, or w/v). The minimum concentration of serum albumin protein during the detecting step may be, for example, at least 0.001, 0.002, 0.003, 0.004, or 0.005% (by weight, or w/v). In one embodiment, the minimum concentration of serum albumin protein is 0.001% (by weight, or w/v). In another embodiment, it is 0.0015 or 0.002% (by weight, or w/v). The concentration of serum albumin protein in the reaction mixture may range from 0.001-0.1%, e.g. 0.002-0.09%, 0.002-0.08%, 0.002-0.07%, 0.002-0.06%, 0.002-0.05%, e.g. 0.003-0.08%, e.g. 0.004-0.07% (by weight, or w/v). In a particular embodiment, the concentration of serum albumin protein may, for example, range from 0.004-0.02% (by weight, or w/v). In a preferred embodiment, the concentration of the serum albumin protein may be 0.01-0.02%, e.g. 0.01 or 0.02% (by weight, or w/v).

The above stated ranges may, for example, apply to a proteinaceous resolving agent selected from BSA, PSA, or HSA, or to another serum protein such as a globulin, or a milk protein.

Alternatively, in another embodiment, a non-proteinaceous resolving agent may be used. For example, the maximum concentration of a non-proteinaceous resolving agent during the nucleic acid product generation (e.g. RCA) reaction may be up to 0.1, 0.2, 0.3, 0.4, 0.5, 0.6, 0.7, 0.8, 0.9, or 1.0% (by weight, or w/v). In one embodiment, the maximum concentration of the non-proteinaceous resolving agent is 0.5% (by weight, or w/v). The minimum concentration of the non-proteinaceous resolving agent during the detecting step may be, for example, at least 0.01, 0.02, 0.03, 0.04, or 0.05% (by weight, or w/v). In one embodiment, the minimum concentration of the non-proteinaceous resolving agent is 0.01% (by weight, or w/v). The concentration of the non-proteinaceous resolving agent in the reaction mixture may range from 0.01-1.0%, for example, 0.01-0.08%, or 0.01-0.5%, e.g. 0.02-0.5, or 0.02-0.4%, e.g. 0.03-0.3%, e.g. 0.04-0.2% (by weight, or w/v).

In one embodiment, a detergent may be used as the resolving agent. For example, the maximum concentration of a detergent during the RCA reaction may be up to 0.1, 0.2, 0.3, 0.4, or 0.5% (by weight, or w/v). In one embodiment, the maximum concentration of the detergent is 0.5% (by weight, or w/v). The minimum concentration of the detergent during the detecting step may be, for example, at least 0.01, 0.02, 0.03, 0.04, or 0.05% (by weight, or w/v). In one embodiment, the minimum concentration of the detergent is 0.01% (by weight, or w/v). The concentration of the detergent in the reaction mixture may range from 0.01-0.5%, e.g. 0.02-0.4%, e.g. 0.03-0.3%, e.g. 0.04-0.2% (by weight, or w/v).

The label may be any detectable label, which may be directly or indirectly signal-giving. Thus, it may be directly or indirectly detectable. A directly detectable label is one that can be directly detected without the use of additional reagents, while an indirectly detectable label is one that is detectable by employing one or more additional reagents, e.g., where the label is a member of a signal producing system made up of two or more components. For example, the label may be spectroscopically or microscopically detectable. Any of the labels used in immunohistochemical techniques may be used. In many embodiments, the label is a directly detectable label, where directly detectable labels of interest include, but are not limited to: fluorescent labels, radioisotopic labels, chemiluminescent labels, coloured labels, particles (e.g. beads, including coloured beads), metallic labels, e.g. gold or silver nanoparticles, metal chelates such as fluorescent or chemiluminescent lanthanide chelates, and the like. Indirectly detectable labels include an enzymatic label, which may generate a detectable, e.g. coloured or chemiluminescent, product. An example of this is horseradish peroxidase (HRP), which is widely used as a label. In the presence of peroxide, HRP oxidizes luminol to 3-aminophthalate, which is an excited product that emits light at 425 nm, allowing it to be detected.

In many embodiments, the label is a fluorescent label. Fluorophores for use as or in such labels are widely known and used in the art. For example, these include but are not limited to: fluorescein, the cyanine dyes, such as Cy3, Cy5, Alexa 555, Bodipy 630/650, and the like.

The label may be introduced into or provided to the nucleic acid product in different ways. For example, it may be incorporated during the synthesis of the nucleic acid product by incorporation of labelled nucleotides. For example, the nucleotide (dNTP) may be fluorescently tagged, e.g. fluorescently tagged CTP (such as Cy3-CTP, Cy5-CTP) etc. In another embodiment, labelled monomers, e.g. labelled HCR monomers, or labelled hybridisation oligonucleotides may be used to construct the nucleic acid product.

Alternatively, non-sequence specific nucleic acid labelling methods may be used to label the nucleic acid products, for example, using DNA binding stains or dyes which are widely known and reported in the literature.

Conveniently, as noted above, the labels may be introduced by means of hybridising labelled oligonucleotides (e.g. detection oligonucleotides) to the nucleic acid products. Thus, as noted above the nucleic acid product may comprise multiple copies of a detection sequence. A detection oligonucleotide which is complementary to, or capable of hybridising to, the detection sequence is employed to provide the label to the nucleic acid product. The detection oligonucleotide may be labelled. Thus, it may carry a label, for example, by means of comprising one or more labelled nucleotides. However, it is not necessary for the detection oligonucleotide to be directly labelled, and it can instead be indirectly labelled, by means of a secondary labelled oligonucleotide which hybridises to the detection oligonucleotide. Thus, the labelled oligonucleotide may be indirectly hybridised to the detection sequence by means of the detection oligonucleotide functioning as an intermediate, or sandwich, oligonucleotide. Such a labelling method is particularly suitable in the case of RCPs.

In the case of a RCP, a detection sequence may readily be included by including a detection sequence (strictly the complementary copy of a detection sequence) in the RCA template circle which is used to generate the RCP. This may, for example, be a padlock probe which upon target binding is circularised to form the RCA template. In the case of a sRCA reaction, a padlock probe may be used to generate the second or further generation RCP, which is ultimately labelled to detect the sRCA product. This is described further below. In the case of a HCR product, a detection sequence may be included in one or more of the HCR monomers. In the case of a hybridisation product, the detection sequence may be included in one or more of the sets of hybridisation probes that are used to generate the product.

A detection sequence is thus simply a sequence by which a nucleic acid product may be detected. In the case of a RCP, a detection sequence may be included in a padlock probe. A complement of the detection sequence will become incorporated into the RCP. The detection sequence may be specific to the padlock probe, and thus to the target sequence, or sequence variant it is desired to detect. Thus, each padlock probe may have a different detection sequence. The detection sequence may be detected to detect or identify which padlock probe was amplified in the RCA, and hence which target sequence was present. Such a protocol may be applied, for instance, in the context of the method for detecting a target sequence variant, where each padlock is provided with a detection sequence specific to a particular variant. The detection sequence may thus be seen as a marker or identification sequence. The term “detection sequence” as used herein includes both the detection sequence as it occurs in the padlock probe, and the complementary copy as it appears in the RCP.

Generally speaking, detection reagents, including labelling reagents may be included in reagent mixes for performing the steps of generating the nucleic acid products, or they may be contacted with the reaction mixture after the generation step.

The resolving agent is included in the mixture comprising the labelled nucleic acid products, which is subjected to the detection step (i.e. in the mixture for detection, or in other words, the detection mixture). This may be achieved in various ways, depending on how the products are generated, and labelled etc. For example, in one embodiment, the resolving agent is contacted with (e.g. added to) the mixture of nucleic acid products. In another embodiment, the resolving agent is present in the reaction mixture when the nucleic acid products are labelled. In another embodiment, the resolving agent is present in the reaction mixture when the nucleic acid products are generated, and remains in the labelled mixture which is detected.

The term “contacting” is used broadly herein to include bringing the reagents) in question into contact. Thus, one may be added to the other and vice versa, or they may each be introduced to each other etc.

For example, the resolving agent may be included in a labelling mix which is contacted with the nucleic acid products after they are generated. Or it may be added separately to the reaction mixture comprising the nucleic acid products, for example, before, together with, or after the labelling mix. It may be contacted with the mixture containing the nucleic acid products in any way, before the labelled nucleic acid products are detected. Conveniently, a labelling mix comprising labelled detection oligonucleotides, or detection oligonucleotides together with complementary labelled oligonucleotides, may comprise the resolving agent. Alternatively or additionally, the resolving agent may be included in the reaction mixture when the nucleic acid product is generated, for example, in the RCA reaction mixture, or in the reaction mixture comprising HCR monomers when the HCR polymer product is generated, or in the reaction mixture when hybridisation probes are hybridised to generate a hybridised nucleic acid product. In the case of a sRCA product, the resolving agent may be included in the reaction mixture for the first and/or second or further (e.g. ultimate) RCA step, or in all of the RCA steps. For example, it may be included in the RCA reagent mix which is used for the RCA reaction.

Thus, in one convenient embodiment, the resolving agent may be included in the reaction mixture for the first RCA of a sRCA reaction. It may be added in excess such that it remains in the reaction mixture, and is present in the detection mixture comprising the labelled sRCA product. It may be included in the reagent mix for the first RCA step, or it may be separately added along with other reagents for the first RCA.

As indicated above, a detection method herein may include the steps of generating the nucleic acid products and labelling them, i.e. generating the mixture which comprises the differently labelled nucleic acid products. The generation steps may be carried out in multiplex. In other words, the different nucleic acid products may be generated in the same reaction mixture. However, it is not precluded that separate reactions are performed in parallel to generate each different nucleic acid product, and the products are then combined, or pooled, to create the reaction mixture.

In a particular embodiment, the nucleic acid products are generated as proxies, or markers, for the detection of target analytes in a detection assay. In particular, they may be generated in the course of a method for detecting target nucleic acid sequences which may occur in one or more target nucleic acid molecules.

The term “detecting” is used broadly herein to include any means of determining the presence of the target nucleic acid sequence in the target nucleic acid molecule. In the present method, the target nucleic acid is detected by detecting the presence or amount of the nucleic acid product which is generated, and can include detecting simply if it is present or not, or any form of measurement of the nucleic acid product. Thus, the nucleic acid product may be detected as the “signal” for the target nucleic acid sequence. Accordingly, detecting the nucleic acid product includes determining, measuring, assessing or assaying the presence or absence or amount or location of the nucleic acid product in any way. The presence of a nucleic acid product (i.e. the confirmation of its presence or amount) is indicative or identificatory of the presence of the target nucleic acid sequence, as the successful generation of the nucleic acid product is ultimately dependent on the presence of the target nucleic acid molecule, and more particularly of the target nucleic acid sequence therein.

Quantitative and qualitative determinations, measurements or assessments are included, including semi-quantitative. Such determinations, measurements or assessments may be relative, for example, when two or more different target nucleic acid sequences, or target molecules, in a sample are being detected, or absolute. Accordingly, in an embodiment, the method may be for quantifying or determining the amount of target nucleic acid sequence which is present. The term “quantifying” when used in the context of quantifying a target nucleic acid sequence(s) in a sample can refer to absolute or to relative quantification. Absolute quantification may be accomplished by inclusion of known concentration(s) of one or more control nucleic acid molecules and/or referencing the detected level of the target nucleic acid sequence with known control nucleic acid molecules or sequences (e.g. through generation of a standard curve). Alternatively, relative quantification can be accomplished by comparison of detected levels or amounts between two or more different target nucleic acid molecules, or different target sequences, to provide a relative quantification of each of the two or more different nucleic acid molecules or sequences, i.e., relative to each other. Thus, as noted above, ratios of target nucleic acid sequences present in sample may be determined. Thus, copy numbers of target nucleic acid molecules, e.g. chromosomes, may be compared.

The methods herein may particularly be used to detect a variant target nucleic acid sequence in a target nucleic acid molecule in a sample. Target nucleic acid sequences may commonly occur in variant forms, for example, allelic variants, or mutant and wild-type sequences, and it may be desirable to detect which variant is present. Accordingly, in an embodiment, the target nucleic acid molecule is an analyte in a sample. However, in another embodiment, the target nucleic acid molecule is not itself the target analyte, but rather is detected as part of an assay to detect another target analyte. Accordingly, in such an embodiment, the target nucleic acid molecule may be a reporter molecule for a target analyte.

The target analyte may thus be any analyte it is desired to detect. As discussed above, in some embodiments, the target nucleic acid molecule of the method herein is the target analyte. In other embodiments, where the target nucleic acid molecule is a reporter, the target analyte may be any analyte it is desired to detect. The analyte may be a nucleic acid, a protein (which term includes peptides and polypeptides), or any other chemical or biological molecule or moiety, including for example carbohydrates, e.g. such as may occur as glycosyl groups on proteins. The target analyte may thus be a modified protein, for example, with a post-translational modification which is detected in an assay for an analyte.

In an embodiment, the target analyte may be a protein or component of a proteinaceous molecule which is detected on the surface of a cell, or vesicle, or other cellular or sub-cellular compartment. For example, extracellular vesicles, or exosomes, may be detected and distinguished by virtue of different proteins present on their surface. Prostasomes have been proposed as biomarkers for prostate cancer, and a particular or selected prostasome or other extracellular vesicle may be detected and distinguished by detecting one or more surface proteins thereon.

The target nucleic acid sequence may be any sequence it is desired to detect or identify. It may be DNA or RNA, or a modified variant thereof. Thus, the nucleic acid may be made up of ribonucleotides and/or deoxyribonucleotides as well as synthetic nucleotides that are capable of participating in Watson-Crick type or analogous base pair interactions. Thus, the nucleic acid may be or may comprise, e.g. bi-sulphite converted DNA, LNA, PNA or any other derivative containing a non-nucleotide backbone.

Typically, the target sequence will be an analyte it is desired to detect, for example, a nucleic acid present in a sample, e.g. in a cell or tissue sample or any biological sample etc. Thus, it may be a naturally occurring sequence, or a derivative or copy or amplicon thereof.

Alternatively, as noted above, the target sequence may instead be a reporter for an analyte of an assay. Reporter nucleic acids may be used or generated in the course of an assay for any analyte, for example a protein or other biological molecule, or small molecule, in a sample. Thus, a reporter nucleic acid may be provided as a tag, or label, for a binding probe for an analyte, and may be detected in order to detect the analyte, for example in an immunoassay, e.g. as in an immunoPCR or immunoRCA reaction. A reporter nucleic acid may be generated in the course of an assay, for example, by a ligation reaction in a proximity ligation assay (PLA), or an extension reaction in a proximity extension assay (PEA), or by a cleavage reaction, or such like. Such a reporter target nucleic acid may therefore be a synthetic or artificial sequence. It may be a linear or a circular or circularised or circularisable molecule.

In an embodiment, the target nucleic acid is a DNA molecule, natural or synthetic. The target nucleic acid molecule may be coding or non-coding DNA, for example, genomic DNA or a sub-fraction thereof, or may be derived from genomic DNA, e.g. a copy or amplicon thereof, or it may be cDNA or a sub-fraction thereof, or an amplicon or copy thereof etc.

In another embodiment, the target nucleic acid molecule is a target RNA molecule. It may be an RNA molecule in a pool of RNA or other nucleic acid molecules, for example, genomic nucleic acids, whether human or from any source, from a transcriptome, or any other nucleic acid (e.g. organelle nucleic acids, i.e. mitochondrial or plastid nucleic acids), whether naturally occurring or synthetic. The target RNA molecule may thus be or may be derived from coding (i.e. pre-mRNA or mRNA) or non-coding RNA sequences (such as tRNA, rRNA, snoRNA, miRNA, siRNA, snRNA, exRNA, piRNA and long ncRNA). In one preferred embodiment, the target nucleic acid molecule is a micro RNA (miRNA). In one embodiment, the target RNA molecule is 16S RNA, for example wherein the 16S RNA is from and identificatory of a microorganism (e.g. a pathogenic microorganism) in a sample. Alternatively, the target RNA molecule may be genomic RNA, e.g. ssRNA or dsRNA of a virus having RNA as its genetic material. Notable such viruses include Ebola, HIV, SARS, SARS-COV2, influenza, hepatitis C, West Nile fever, polio and measles. Accordingly, the target RNA molecule may be positive sense RNA, negative sense RNA, or double-stranded RNA from a viral genome, or positive-sense RNA from a retroviral RNA genome.

Where the target molecule is an RNA molecule, the method may comprise a preliminary step of generating a cDNA copy of the target RNA molecule.

Methods for generating a nucleic acid product as used herein are well known in the art, and widely described in the literature, as are detection methods employing them.

Thus, RCA is widely known as an amplification technique, and many detection assays have been proposed and described using RCA to generate a detectable product. As noted above, WO 2015/071445, for example, describes the basic principles of a sRCA reaction and its use in detection assays in general, and its disclosure is incorporated herein by reference.

The template circle for a RCA reaction, or for the first RCA reaction of a sRCA reaction, may be produced by the circularisation of a probe for a target nucleic acid sequence, notably a padlock probe, according to principles well-known in the art. Padlock probes may take many forms, and may be provided in 1-part form, or multi-part (e.g. 2-part) form. They include gap-fill padlock probes (also known as molecular inversion probes (MIPs)). The RCA template may alternatively be a pre-formed circle, which forms part of a target-specific probe (e.g. is hybridised to a target-specific probe, or to a nucleic acid part or domain thereof), or is used together with a target specific probe, such as in immunoRCA reactions, for example. Analogously, it may be a circularisable oligonucleotide which is ligated to form a circle during the course of the assay reactions. Thus, an RCP (as a representative nucleic acid product) may be a product of an immunoRCA reaction or any type of detection reaction which comprises a RCA step, for example, a proximity probe assay in which a circular nucleic acid molecule is generated-see the Duolink™ PLA of SigmaAldrich for example, and the modified PLA which uses so-called Unfold proximity probes, which comprise hairpins which are opened, or unfolded, by cleavage to release nucleic acid domains which may be circularised to form a RCA template (see Klaesson et al, 2018, Scientific Reports 8, 5400).

In more detail, a padlock probe may alternatively be defined as a circularisable probe. The use of padlock or circularisable probes is well known in the art, including in the context of RCA reactions. A circularisable probe comprises one or more linear oligonucleotides which may be ligated together to form a circle. Padlock probes are well known and widely used and are well-reported and described in the literature. Thus, the principles of padlock probing are well understood and the design and use of padlock probes is known and described in the art. A padlock probe is typically a linear circularisable oligonucleotide which hybridises to its target nucleic acid sequence or molecule in a manner which brings 5′ and 3′ ligatable ends of the probe into juxtaposition for ligation together, either directly or indirectly, with a gap in between. By ligating the hybridised 5′ and 3′ ends of the probe, the probe is circularised. It is understood that for circularisation (ligation) to occur, the ligatable 5′ end of the padlock probe has a free 5′ phosphate group.

To allow the juxtaposition of the ends of the padlock probe for ligation, the padlock probe is designed to have the target-binding sites at or near its 5′ and 3′ ends. That is, the regions of complementarity which allow binding of the padlock probe to its target lie at or near the ends of the padlock probe.

To allow ligation, the 3′ and 5′ ends which are to be ligated (the “ligatable” 3′ and 5′ ends) are hybridised to the target sequence in the first RCP, which acts as the ligation template. The ligatable ends of a padlock probe may be brought into juxtaposition for ligation in various ways, depending on the probe design. Where the target-binding sites are located at the ends of the padlock probe, the binding of the padlock probe may bring the ends into said juxtaposition. Where the complementary binding sites in the target molecule or sequence lie directly adjacent (or contiguous) to one another, the ends of the padlock probe will hybridise directly adjacent to each other (i.e. with no gap) and may be ligated to each other directly. Thus, in this case, the ligatable ends of the probe are provided by the actual ends of the probe. However, in an alternative configuration, the padlock probe is a gap-fill padlock probe, and hence the binding sites at the ends of the padlock probe do not hybridise to adjacent binding sites, but rather to non-adjacent (non-contiguous) binding sites in the target sequence. In such an arrangement, the 5′ ligatable end of the probe is provided by the actual 5′ end of the probe. However, the ligatable 3′ end of the probe is generated by extension of the hybridised 3′ end of the probe, using the target sequence as an extension template to fill the gap between the hybridised ends of the probe. The extension reaction brings the extended 3′ end of the probe into juxtaposition for ligation. In this case, the ligatable 3′ end of the probe is thus the extended 3′ end of the probe.

Padlock probes may be provided in 2 or more parts that are ligated together. This may involve the provision of an additional ligation template, for example, in the case of a 2-part probe, where each part comprises only one target-binding region, and the other end of each part hybridises to a common ligation template. In another embodiment, a 2-part padlock may take the form of a “connector” oligonucleotide with two target-binding regions at or near the 5′ and 3′ ends respectively, which hybridise to the target with a gap in between them, and a gap oligonucleotide which hybridises in the gap between the ends. The gap oligonucleotide may partially or fully fill the gap.

Alternatively, the RCA template (or first RCA template of a sRCA) used to generate the RCP may be a circularised target nucleic acid sequence, or an amplicon, e.g. PCR or other copy thereof. A target nucleic acid molecule, or an amplicon of the target sequence may be circularised using a ligation template which hybridises to the ends of the molecule. Circularisation adaptors, or so-called “Selectors”, for circularisation of target nucleic acid molecules are described in WO 99/049079, WO 2003/012119 and WO 2005/070630.

HCR is also well known and widely described, and HCR has been used as a signal amplification method in detection assays. A proximity assay based on HCR is described in WO 2015/118029, which together with the other documents referenced above, is incorporated herein by reference.

A detection method based on sRCA using gap-fill padlock probes is described in WO2022/117769, and is particularly suited to the detection of variant target nucleic acid sequences. In an embodiment, the methods herein are particularly suited for use in the context of such a method. In the method, a target-specific first padlock probe is used to “capture” a target sequence by hybridising to the target nucleic acid molecule and generating a complementary copy of the target sequence by a gap-fill extension reaction. The extended 3′ end of the first padlock probe is ligated to the 5′ end of the padlock probe to circularise it, and thereby generates the first RCA template. The circularised first padlock probe containing the complementary copy is then amplified by RCA to generate a first RCA product containing multiple copies of the target sequence. The resulting first RCA product is then probed with a further, second, padlock probe, specific for the target sequence. The circularised second padlock probes are subjected to a further, second RCA reaction, which is used to generate second RCA products attached to the first, and the resulting sRCA product is detected to detect the target sequence. Thus, the resulting sRCA product may represent a nucleic acid product in the methods herein.

In an alternative version of such a sRCA-based assay, rather than using a padlock probe to generate a copy of the target sequence, amplicons of the target sequence may be prepared, conveniently by a PCR reaction, although any amplification method may be used. The amplicons are then circularised by ligation, conveniently but not necessarily using a ligation template, to generate a first RCA template. This is amplified by RCA, and thereafter the detection method continues as above to generate a sRCA reaction product which is detected. The resulting sRCA product represents a further example of a nucleic acid product in the methods herein.

Accordingly, in a particular embodiment, the detection method herein for detecting two or more target nucleic acid sequences may comprise:
(a) providing a reaction mixture comprising amplicons of the target nucleic sequences;
(b) ligating the 5′ and 3′ ends of single-stranded amplicons from (a) to circularise them;
(c) performing first RCA reactions using the circularised amplicons as first RCA templates to generate first RCA products (RCPs) comprising multiple repeats of a complementary copy of the target nucleic acid sequence in the amplicons;
(d) contacting the first RCPs with two or more different padlock probes specific for the two or more target nucleic acid sequences and allowing the probes to hybridise to the complementary copies in the multiple repeats;
(e) directly or indirectly ligating the ends of the hybridised padlock probes which have hybridised to their target sequence complements (i.e. to the nucleic acid sequence complements which are the targets of, or which correspond to the respective padlock probes) to circularise the hybridised padlock probes;
(f) performing second RCA reactions using the circularised padlock probes as second RCA templates to generate second RCPs containing multiple repeat complementary copies of the circularised padlock probes, wherein different second RCPs are generated for each target nucleic acid sequence to be detected in the reaction mixture;
(g) providing each of the second RCPs of (f) with multiple copies of one or more labels, wherein each different second RCP is differently labelled and can be distinguished from another second RCP, and wherein the labels are provided during the generation of the second RCPs in (f) or after, thereby generating a detection mixture comprising two or more differently labelled second RCPs, wherein a resolving agent is included in the detection mixture to facilitate resolution of the differently labelled RCPs, wherein said resolving agent acts to inhibit interaction between different labels;
(h) detecting the labelled second RCPs in the detection reaction mixture, thereby detecting the target nucleic acid sequences.

In step (e), correctly hybridised padlock probes are ligated, that is those that have correctly and specifically hybridised to the complements of the target sequences that they are designed to detect (i.e. the padlock probes which have hybridised to their respective target sequence complements).

It will thus be understood that in step (d), the padlock probes will hybridise to their intended target (i.e. specifically), where that target sequence is present.

Whilst padlock probes may be designed with target-specific binding regions which are specific for a particular target sequence, it will be understood that non-specific hybridisation may occur, particularly in the case of variant target sequences which are similar in sequence (e.g. where the variants are single nucleotide variants, such as SNPs, and such like). However, the high specificity of padlock probes arises from the failure of such non-specifically hybridised padlock probes to be ligated (and hence any such non-ligated padlock probes would not be amplified by the subsequent RCA step). Thus, in the second ligation step of the detection method above, the padlock probes which have hybridised to their target sequence complements are ligated (that is the padlock probes which have correctly hybridised to the complement of the target sequence they are intended to detect, or in other words, their corresponding or cognate, or respective target sequence).

In an embodiment, the amplicons in (a) are PCR amplicons. This includes any variant of PCR, including asymmetric PCR, for example.

In an embodiment, the method may include the step of performing the amplification reaction, e.g. PCR, to generate the amplicons. The amplicons of individual target sequences may be prepared together in multiplex, in a single reaction mixture, or they may be separately prepared in parallel, and then pooled or mixed to provide the reaction mixture of (a).

The improvement in detection afforded by the use of a resolving agent according to the methods herein is particularly advantageous in the context of performing detection methods such as those discussed above in a single reaction vessel. Such “single-vessel” methods are advantageous from the point of view of ease of operation and automation but require “clean” reactions where any potential interference from or between reaction components or other materials present in the reaction mixture is reduced or avoided.

sRCA methods such as those described above require a first RCA step, and at least a second RCA step, templated by the circularised padlock probe. The method may comprise further RCA steps, to generate a third, or further generation RCA product, using third, or fourth padlock probes, and so on, each targeting the target nucleic acid sequence. The final generation RCA product may be detected.

To carry out the step of detecting the nucleic acid products the reaction mixture, or a part of aliquot thereof, may be removed from the vessel in which the reaction was performed, and moved to a detection instrument, or for further processing before detection. Alternatively, the detection may take place directly, for example, where a coloured reaction product may be directly observed in the reaction vessel (e.g. where coloured beads are used to label the reaction product, for example in a tube).

The method may be carried out in heterogenous or homogenous formats. That is, it may be performed on a solid phase (or support), or in solution or suspension (i.e. without a solid phase or support), or indeed both, since a solid phase may be introduced at a later stage.

The format of the method may be selected based on the nature of the sample, or the target nucleic acid molecule, or the desired readout or detection technology used. In an embodiment, the method is an in-solution method, that is, a method performed in a liquid phase contained in a reaction vessel.

The target nucleic acid molecule may be present within a sample, or obtained from a sample. The sample may be any sample which contains any amount of nucleic acid, from any source or of any origin, in which it is desired to detect a target nucleic acid sequence in a target nucleic acid molecule. A sample may thus be any clinical or non-clinical sample, and may be any biological, clinical or environmental sample in which the target nucleic acid molecule may occur.

The sample may be any sample which contains a target nucleic acid molecule, and includes both natural and synthetic samples, that is, materials which occur naturally or preparations which have been made. Naturally occurring samples may be treated or processed before being subjected to the methods herein. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way e.g. for storage.

Representative samples thus include any material which may contain a target nucleic acid molecule, including for example foods and allied products, clinical and environmental samples. The sample may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue green algae, fungi, bacteria, protozoa etc., or a virus. The cells may be, for example, human cells, avian cells, reptile cells etc., without limitation.

Representative samples thus include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The sample may be pre-treated in any convenient or desired way to prepare for use in the method, for example by cell lysis or purification, isolation of the nucleic acid, etc.

In one embodiment, the sample comprises microbial cells or viruses which have been isolated from a clinical sample or from a culture of a clinical sample. In such a sample, the target nucleic acid molecule may be a nucleotide sequence present in a microbial cell, e.g. a nucleotide sequence which is characteristic for, or discriminatory or identificatory of a microbial cell or virus, at any level, e.g. at type, group, class, genus, species or strain level.

In another embodiment, the sample may contain cell-free DNA. The sample may be a sample such as plasma or serum which directly contains cell-free DNA, or the cell-free DNA may be isolated.

Since the target nucleic acid molecule need not itself be the target analyte of the assay, but can be a reporter molecule used or generated in the course of an assay for any desired analyte, the sample need not be a sample which naturally contains nucleic acid, or a source of nucleic acid (e.g. a cell or virus, or biological or clinical material etc.). As indicated above, the sample may be a synthetic or artificial sample. It may accordingly be a sample which has been subjected to a detection assay for an analyte in which a target nucleic has been generated, or to which a target nucleic acid molecule has been added. It may be a reaction mixture, or a reaction product, for example, the product resulting from an immunoassay to detect a target analyte, e.g. an immunoPCR, immunoRCA, or proximity assay (e.g. proximity ligation assay (PLA) or proximity extension assay (PEA).

The term “hybridisation” or “hybridises” as used herein refers to the formation of a duplex between nucleotide sequences which are sufficiently complementary to form duplexes via Watson-Crick base pairing, or any analogous base-pair interactions. Two nucleotide sequences are “complementary” to one another when those molecules share base pair organization homology. Hence, a region of complementarity in a molecule or probe or sequence refers to a portion of that molecule or probe or sequence that is capable of forming a duplex. Hybridisation does not require 100% complementarity between the sequences, and hence regions of complementarity to one another do not require the sequences to be fully complementary, although this is not excluded. Thus, the regions of complementarity may contain one or more mismatches. Accordingly, “complementary”, as used herein, means “functionally complementary”, i.e. a level of complementarity sufficient to mediate a productive hybridisation, which encompasses degrees of complementarity less than 100%. The degree of mismatch tolerated can be controlled by suitable adjustment of the hybridisation conditions. Those skilled in the art of nucleic acid technology can determine duplex stability empirically considering a number of variables including, for example, the length and base pair composition of the respective molecules or probe oligonucleotides, ionic strength, and incidence of mismatched base pairs, following the guidance provided by the art. Thus, the design of appropriate probes, or ligation templates or primers etc. for any of the reaction steps described herein, and binding regions thereof, and the conditions under which they hybridise to their respective targets is well within the routine skill of the person skilled in the art.

A region of complementarity, such as, for example, to a target sequence in the binding region of a padlock probe, or between a detection sequence and a detection probe, or a RCA primer to the circularised padlock probe etc., may be at least 6 nucleotides long, to ensure specificity of binding, or more particularly at least 7, 8, 9 or 10 nucleotides long. The upper limit of length of the region is not critical, but may, for example, be up to 50, 40, 35, 30, 25, 20 or 15 nucleotides. A complementary region may thus have a length in a range between any one of the lower length limits and upper length limits set out above. In the case of a padlock probe, the length of an individual target-binding region may be in the lower ranges, so that the total length of the two binding regions when hybridised to their target is within the upper ranges. For example, an individual target binding region may be 8-15, e.g. 10-12 nucleotides, so that the total hybridised length is 16-30 nucleotides long, e.g. 20-24. It may be desirable, within the constraints of conformation of the probes, and spacing of the domains, and desired or favoured hybridisations, to minimise the total length of a padlock probe to minimise the size of the circle which is subjected to RCA, and hence to minimise the lengths of the complementary regions where possible.

The labelled nucleic acid products may be detected using any convenient protocol or detection modality. This may depend on the target sequence to be detected, the purpose of the method, and/or the specific details of the procedures employed in the method, and the labels used.

For example, the labelled products may be detected using any of the well-established methods for analysis of nucleic acid molecules known from the literature including mass spectrometry, CyTOF, microscopy, real-time PCR, fluorescent probes, microarray, colorimetric analysis such as ELISA, flow cytometry, mass spectrometry, or by turbidometric, magnetic, particle counting, electric, surface sensing, weight-based detection techniques.

Depending on the level of multiplexing, combinatorial labelling methods may be used, according to techniques well known in the art. For example, the large number of repeated sequences in the sRCA products can enable distinction amongst large numbers of such products via ratio labelling with fluorescent or other spectrophotometrically detectable probes. Such ratio-labelled detection probes may be used during flow cytometry, or microscopic detection techniques, e.g. imaging, to detect large numbers of sequences, e.g. the combination of at least two fluorophores at different ratios can lead to generation of multiple populations of fluorescent labels. For example, it has been found that using combinations of two fluorophores at different ratios, 7 different populations can be created. This may be expanded using 3- or 4-colour combinations.

Although various detection modalities may be employed, conveniently the labelled nucleic acid products may be detected by microscopy or flow cytometry. In both cases directly or indirectly labelled detection oligonucleotides may be used, for example, with fluorescent labels which may readily be detected. In particular, in a microscopy-based method, the labelled products (e.g. RCPs) may be detected by imaging.

The use of such detection techniques advantageously allow the nucleic acid products to be digitally recorded. Indeed, since the degree of signal amplification afforded by the high molecular weight nucleic acid products/NAAs in the methods herein allows them to be visualised, and they may be detected by a camera or any device including a camera, such as a mobile phone.

To detect products generated in a homogenous format, they may be captured or brought down to a solid support, or surface, to facilitate imaging, or microscopic detection more generally. Particularly in the case of sRCA products, a second RCP, being a second generation RCA product, is larger and heavier and hence readily amenable to bringing down to a surface by centrifugation. Thus, for example, tubes or plates may readily be spun to bring second RCPs down to the bottom of the tube or of a well for detection by microscopy, and particularly imaging.

The method will now be described in more detail with reference to the following Figures and non-limiting examples.

### DESCRIPTION OF FIGURES

FIG. 1: schematic illustration of the method for generation of SuperRCA amplification products. A) DNA sequences of interest in a sample are amplified by PCR. B) Amplified strands are converted to single-stranded DNA circles via templated ligation of their 5′ and 3′ ends. C) Oligonucleotides that template the circularisation reactions next serve as primers for RCA reactions. D) The RCA products are then interrogated with padlock probes specific for mutant or wildtype sequences. E) Ligated padlock probes, wound around the RCA products, thereafter template secondary RCA reactions, primed by an added oligonucleotide. F) For each starting DNA circle, the reaction gives rise to large clusters of mainly single-stranded DNA objects, called SuperRCA products. Up to a million fluorescence-labelled hybridisation probes can bind each of the mutant- or wildtype-specific products, allowing efficient counting via e.g. standard flow cytometry.

FIG. 2: Flow cytometry readout data of single tube SuperRCA assays revealing (A) the IDH2 p.R140Q mutation at 1% spike-in with BSA in the reaction and (B) the IDH2 p.R140Q mutation at 1% spike-in without BSA in the reaction.

FIG. 3: Flow cytometry readout data of single tube SuperRCA assays revealing the IDH2 R172K mutation at (A) 1.024%, (B) 0.064%, (C) 0.001%, and (D) wild-type samples which are negative for the IDH2 R172K mutation.

FIG. 4: Analysis of four-fold serial dilutions of genomic DNA from cells with IDH2 p.R172K point mutation using the SuperRCA procedure or ddPCR. Genomic DNA samples with mutant genomic DNA serially diluted in wildtype genomic DNA were each divided into two portions and analyzed with the SuperRCA or ddPCR assays. 330 ng DNA, corresponding to 100,000 human haploid genomes, were analyzed per sample for the SuperRCA assay, while 66 ng, or 20,000 human haploid genome equivalents, were used for ddPCR, according to the manufacturer's instructions. Each data point was calculated as means of three biological replicates, the limit of detection (LoD) for ddPCR and SuperRCA assay for each analyte was calculated respectively (LoD=Mean (Wildtype)+3×SD (wildtype)) and presented in the figures with dash lines.

FIG. 5: Patient UPN126 was diagnosed with primary myelofibrosis two years prior to AML diagnosis. The bone marrow sample at AML diagnosis (day 0) revealed high levels of the IDH2 p.R140Q mutation as recorded via NGS, SuperRCA and ddPCR. Retrospective analysis of the initial bone marrow sample in the myelofibrosis stage was negative for the IDH2 p.R140Q mutation by SuperRCA and ddPCR assays. SCT was performed on day 139, at which point considerable levels of the mutation remained. The IDH2 p.R140Q mutation was detected at low levels by the SuperRCA assay approximately three months post SCT, whereas ddPCR failed to identify the detectable levels of the mutation at that timepoint. All subsequent samples have been negative in blood and bone marrow by SuperRCA and ddPCR analysis, indicating a complete remission.

FIG. 6: Flow cytometry readout data of single tube SuperRCA assays revealing (A) the R132H mutation at 1.024% spike-in with 0.002% BSA (by weight, or w/v) in the assay; (B) the R132H mutation at 1.024% spike-in with 0.005% BSA (by weight, or w/v) in the assay; (C) the R132H mutation at 1.024% spike-in with 0.01% BSA (by weight, or w/v) in the assay; and (D) the R132H mutation at 1.024% spike-in with 0.02% BSA (by weight, or w/v) in the assay.

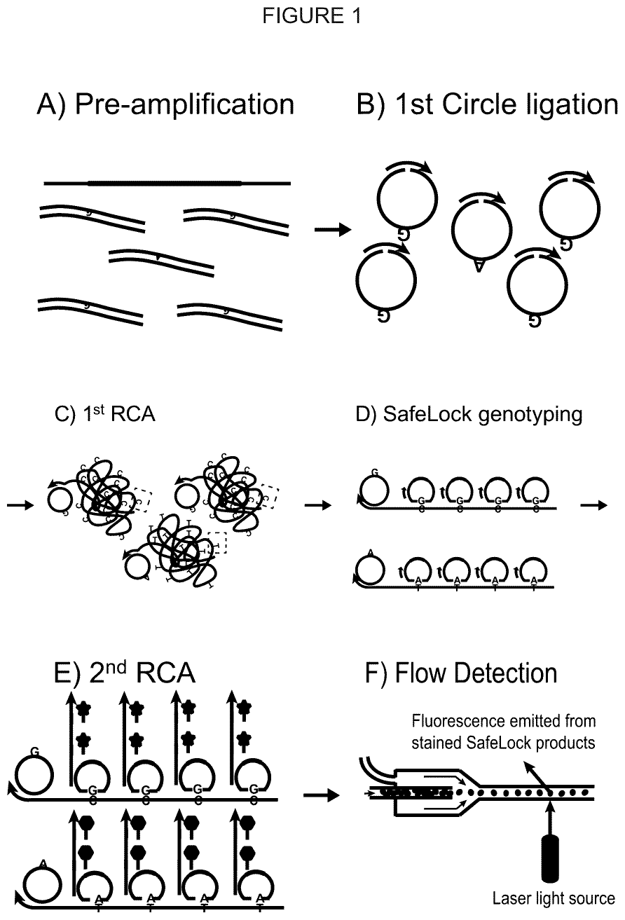

### EXAMPLES

### Materials and Methods

### Extraction of Genomic DNA.

DNA was extracted from BM cells or of whole blood using the QIAamp DNA Blood mini kit (Qiagen cat.51104) and eluted in 50 μL elution buffer.

### PCR Based Library Prep: Tube a

### High Fidelity PCR Pre-Amplification.

Sequences of interest in genomic DNA were amplified with SuperFi DNA polymerase (Thermo Scientific) in 25 μL PCR reactions containing 1× SuperFi buffer, 0.2 mM dNTP, 100 nM Fwd/Rev PCR primers, 330 ng gDNA and 0.0005 U/μL SuperFi DNA polymerase. The PCR program was as follows: 98° C. for 30 sec, 10 cycles of 98° C. for 15 sec, 62° C. for 120 sec, and a final elongation at 72° C. for 5 min.

### SuperRCA Assay: Tube B

### Primer and PCR Polymerase Clean Up:

1 μL per target from the PCR based library prep was mixed with 20 μL clean-up solution containing 1× SuperRCA buffer (Rarity Bioscience AB), 0.125 μL Exol (Thermo Fisher Inc.), and 0.0006 U/μL Thermoliable Proteinase K. The mixture was incubated at 37° C. for 10 min, followed by 55° C. for 10 min.

### Ligase-Mediated Circularisation of One Strand of PCR Products.

20 μl ligation solution containing 1× SuperRCA buffer (Rarity Bioscience), 100 nM of ligation template, complementary to both ends of one strand of the amplification products, 0.5 mM NAD (Sigma) and 2 U Ampligase (Lucigen) were added into the clean-up solution containing amplified PCR products. The mixtures were incubated at 95° C. for 1 min, followed by 58° C. for 30 min.

### Target Sequence Amplification by a First RCA.

Circularised strands of PCR products containing target nucleotide positions were amplified by RCA. 5 μL of 1× SuperRCA buffer (Rarity Bioscience), 1.8 mM dNTP (Invitrogen), 2.5 U Phi29 polymerase (New England Biolabs) and BSA at a concentration of 1.8 μg/μL were added to the circularised products. The resulting reaction mixture comprises BSA in excess. The reactions were incubated at 37° C. for 30 min, then 65° C. for 10 min.

### Genotyping of RCA Products Via Padlock Probe Ligation.

Padlock probes were hybridised to first-generation RCA products and ligated in a sequence-specific manner, by adding 5 μL ligation mix containing 1× SuperRCA buffer (Rarity Bioscience), 3 mM NAD (Sigma), 2.5 U Ampligase (Lucigen), and 60 nM genotyping padlock probe pairs to the reaction mixtures, incubating at 55° C. for 30 min.

### Digestion of the Non-Reacted Genotyping Probes.

5 μL clean-up solution containing 1× SuperRCA buffer (Rarity Bioscience AB), 1.2 μM primer and 1 U/μL Lambda exo was added into the reaction mixture and incubated at 37° C. for 15 min then at 75° C. for 20 min.

### Secondary RCA Templated by Padlock Probes Bound to Primary RCA Products.

5 μL RCA mixture containing 1× SuperRCA buffer (Rarity Bioscience), 0.6 mM dNTPs and 6 U Phi29 DNA polymerase (New England Biolabs) was added to the mixture and the reactions were incubated at 37° C. for 30 min.

### Digital Recording of SuperRCA Products by Flow Cytometry.

The final reaction mixtures containing SuperRCA products were diluted into hybridisation buffer containing 100 nM fluorophore-labelled oligonucleotide probes specific for the different SuperRCA products, in 1× SuperRCA buffer (Rarity Bioscience) to a final volume of 250 μL. The solutions were applied onto the CytoFlex flow cytometer (Beckman Coulter) and SuperRCA products were counted at ‘Medium’ speed (30 μL/minute) for 150 seconds per sample.

### Example 1

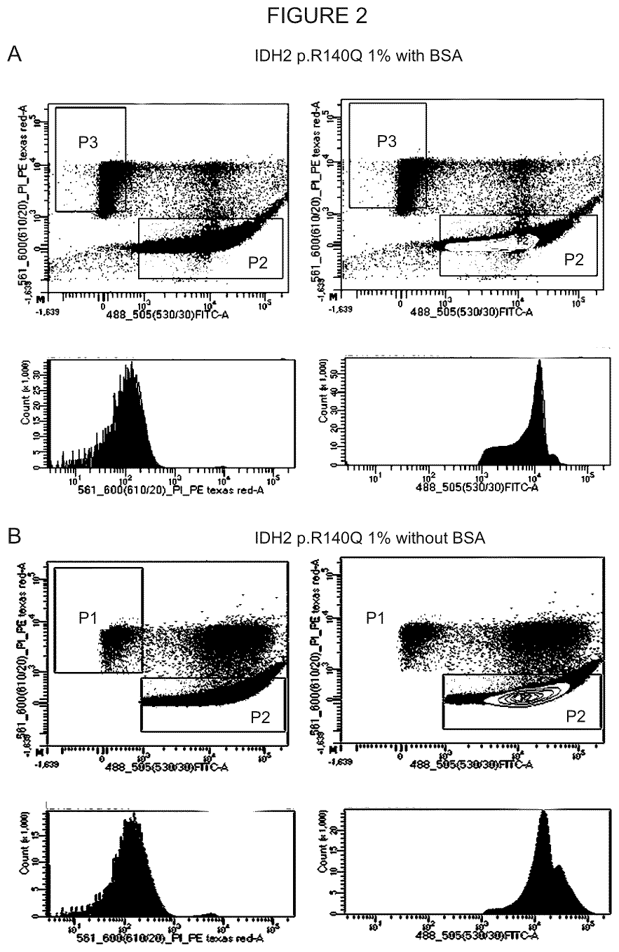

The presence of BSA in the SuperRCA assay helps to resolve the mutants and wild-type population by preventing them from clustering together in the solution; the absence of BSA would otherwise result in an increased population in the top-right corner of the graph (double positive population). The presence of BSA (FIG. 2A) helps to place more mutant events into the gate, increase the detected mutant frequency, and reduce the double positive events in the top-right corner. The reaction without BSA (FIG. 2B) results in underestimated mutant allele frequency and creates a higher percentage of double positive events that do not fall into either of the gates. The number of events detected by flow cytometry for each reaction are displayed in Tables 1 to 2 below. P3 and P1 are the probes detecting mutant events, and P2 is the probe detecting wildtype events. It can be seen that there are greater percentages of all events captured by a single probe in Table 1 than in Table 2, which represents reduced clustering and thus fewer double positives.

Tables 1-2 present the raw data from the flow cytometry readouts revealing, respectively, 1% of the IDH2 p.R140Q mutation with BSA, and 1% of the IDH2 p.R140Q mutation without BSA.

TABLE 1
(FIG. 2A)
IDH2 p.R140Q 1% with BSA

| Population | #Events | % Parent | Mean | Mean |
|---|---|---|---|---|
| All Events | 1,318,680 | #### | 8,828 | 179 |
| P2 | 1,300,338 | 98.6 | 8,754 | 105 |
| P3 | 11,122 | 0.8 | 136 | 6,124 |

TABLE 2
(FIG. 2B)
IDH2 p.R40Q 1% without BSA

| Population | #Events | % Parent | Mean | Mean |
|---|---|---|---|---|
| All Events | 757,094 | #### | 20,273 | 235 |
| P2 | 740,005 | 97.7 | 19,783 | 150 |
| P1 | 3,358 | 0.4 | 295 | 4,585 |

### Example 2

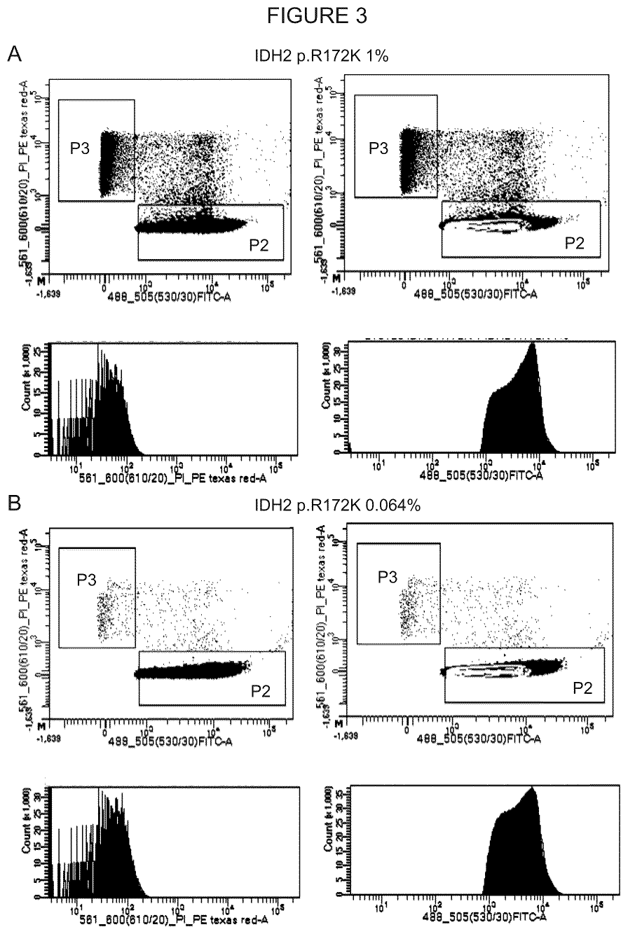

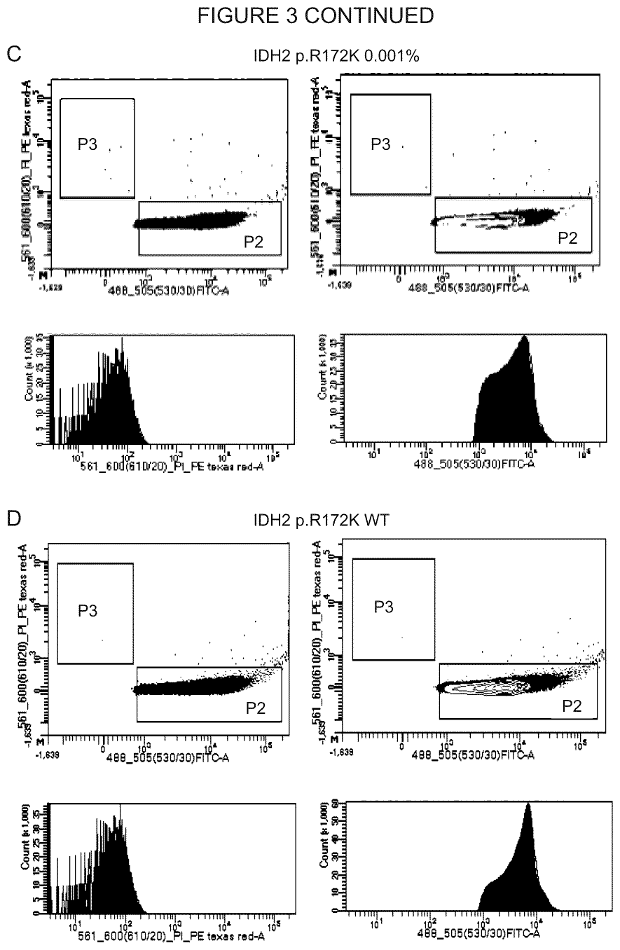

The single tube SuperRCA assay was used to analyse samples with varying levels of the IDH2 p.R172K mutation spiked into the sample, ranging from 1% to 0% (comprising a wildtype sample). The SuperRCA products were detected using BD Fortessa flow cytometer (FIG. 3). The number of events detected by flow cytometry for each sample with varying abundances of IDH2 p.R172K mutation are displayed in Tables 3 to 6 below. P3 is the probe detecting mutant events, and P2 is the probe detecting wildtype events. As can be seen from the flow diagrams in FIG. 3 and the data in Tables 3 to 6, the signal from P3 decreases as the level of spike-in mutations in the samples decrease from 1% to 0%, and even at a very low level of mutation of 0.001%, the SuperRCA assay was still able to detect the mutant signal (FIG. 3C), demonstrating the sensitivity of the assay.

Tables 3-6 present the raw data from the flow cytometry readouts revealing, respectively, 1%, 0.064%, 0.001% and 0% (WT) of the IDH2 R172K mutation.

TABLE 3
(FIG. 3A)
IDH2 p.R172K 1%

| Population | #Events | % Parent | Mean | Mean |
|---|---|---|---|---|
| All Events | 1,326,746 | #### | 4,811 | 59 |
| P2 | 1,317,274 | 99.3 | 4,827 | 22 |
| P3 | 6,502 | 0.5 | 62 | 5,652 |

TABLE 4
(FIG. 3B)
IDH2 p.R172K 0.064%

| Population | #Events | % Parent | Mean | Mean |
|---|---|---|---|---|
| All Events | 1,675,508 | #### | 4,289 | 32 |
| P2 | 1,672,568 | 99.8 | 4,291 | 29 |
| P3 | 393 | 0.0 | 55 | 5,315 |

TABLE 5
(FIG. 3C)
IDH2 p.R172K 0.001%

| Population | #Events | % Parent | Mean | Mean |
|---|---|---|---|---|
| All Events | 1,668,478 | #### | 5,086 | 35 |
| P2 | 1,667,261 | 99.9 | 5,083 | 35 |
| P3 | 9 | 0.0 | 136 | 4,300 |

TABLE 6
(FIG. 3D)
IDH2 p.R172K WT (0%)

| Population | #Events | % Parent | Mean | Mean |
|---|---|---|---|---|
| All Events | 1,774,964 | #### | 5,530 | 36 |
| P2 | 1,774,369 | 100.0 | 5,521 | 35 |
| P3 | 1 | 0.0 | −42 | 2,041 |

### Example 3: Detection of the Ultralow Frequency AML Point Mutation IDH2 p.R172K Using SuperRCA or ddPCR Assays

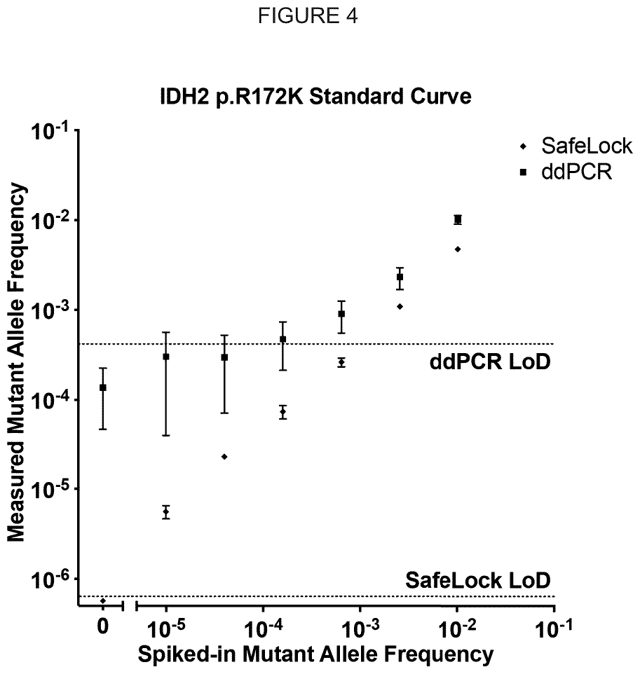

The PCR amplicons targeting DNA sequences containing IDH2 p.R172K mutations were applied in the spike-in DNA samples for the SuperRCA pre-amplification step. Then the SuperRCA protocol was applied on the amplified PCR products to estimate the mutant allele frequency in each spike-in sample, and the SuperRCA products were analyzed with Cytoflex flowcytometers. For the corresponding ddPCR assay, the same spike-in DNA samples were used in the Bio-Rad ddPCR platform with 66 ng per replicates and performed in duplicates. The results as shown in FIG. 4 demonstrate that the SuperRCA assay has a lower limit of detection (LoD) and successfully detected mutant DNA in up to 100,000-fold excesses of normal DNA in comparison to ddPCR, which revealed substantially greater variability and insufficient sensitivity to detect very low frequency mutations.

### Example 4: Monitoring Disease Progression in an AML Patient with the IDH2 p.R140Q Mutation Using SuperRCA, NGS and ddPCR Assays

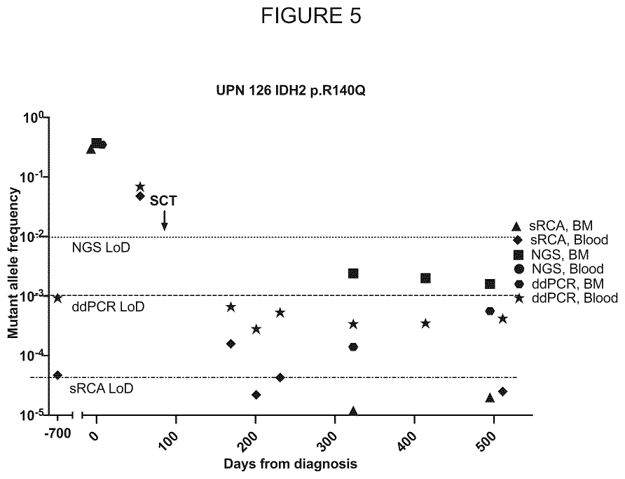

Samples from an AML patient (UPN126) with the IDH2 p.R140Q mutation were analyzed at several time points using SuperRCA, NGS and ddPCR assays to explore the suitability of SuperRCA probing for monitoring the course of disease (FIG. 5). Patient UPN126 was previously diagnosed with primary myelofibrosis associated with a JAK2-mutation, which had transformed to a secondary AML two years after the initial diagnosis. NGS at AML diagnosis, carried out by the clinical collaborators at the Uppsala University Hospital with Trusight NGS panel and sequenced with Illumina MiSeq platform, revealed the presence of an IDH2 p.R140Q mutation alongside the previously known JAK2-mutation. Retrospective analysis of the initial bone marrow sample in the myelofibrosis stage was negative for the IDH2 p.R140Q mutation when analyzed by both SuperRCA and ddPCR assays, indicating a clonal evolution upon AML transformation. The PCR amplicons targeting DNA sequences containing IDH2 p.R140Q mutations were applied in the patient DNA samples for the SuperRCA pre-amplification step. Then the SuperRCA protocol was applied on the amplified PCR products to estimate the mutant allele frequency in each time point sample, and the SuperRCA products were analyzed with Cytoflex flow cytometer. For the corresponding ddPCR assay, the same DNA samples was used in the Bio-Rad ddPCR platform with 66 ng per replicates and performed in duplicates using IDH2 p.R140Q ddPCR assay pre-validated by Bio-Rad. The patient achieved a CR after the first course of intensive chemotherapy but remained MRD-positive at approximately 2% (measured by flow cytometry of bone marrow cells with CD34, CD117, CD13 and DR⁺ markers) after 3 courses of intensive chemotherapy. An allogeneic SCT was performed on day 139. The patient was subsequently monitored by analyzing consecutive blood and BM samples through ddPCR and NGS and all six samples collected post-SCT revealed undetectable levels of the IDH2 p.R140Q mutation by both assays, indicating that this patient has remained in remission with a follow up of more than two years post SCT. The blood sample collected 3 months post SCT was positive by SuperRCA but under the detection limit for ddPCR (FIG. 5), demonstrating the higher level of sensitivity of the SuperRCA assay.

### Example 5: Establishing Effective Concentrations of BSA as a Resolving Agent

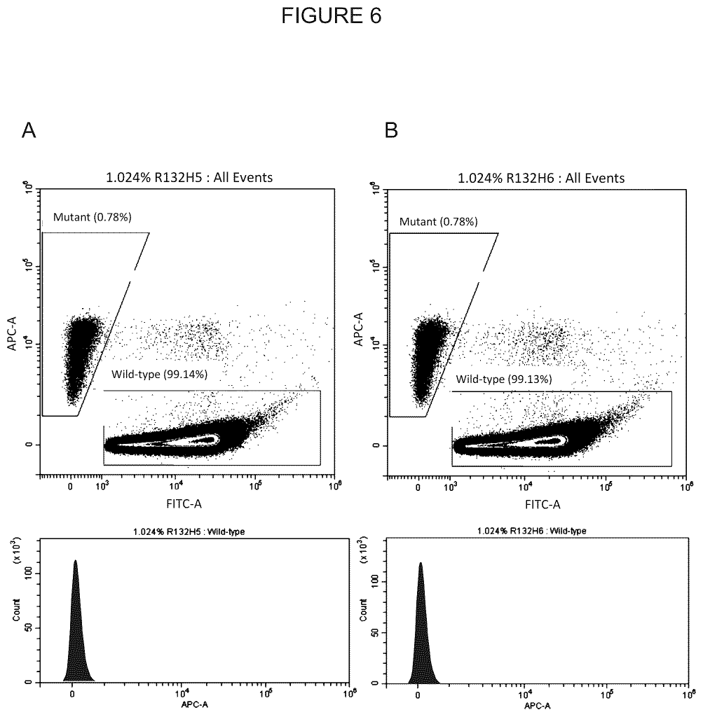

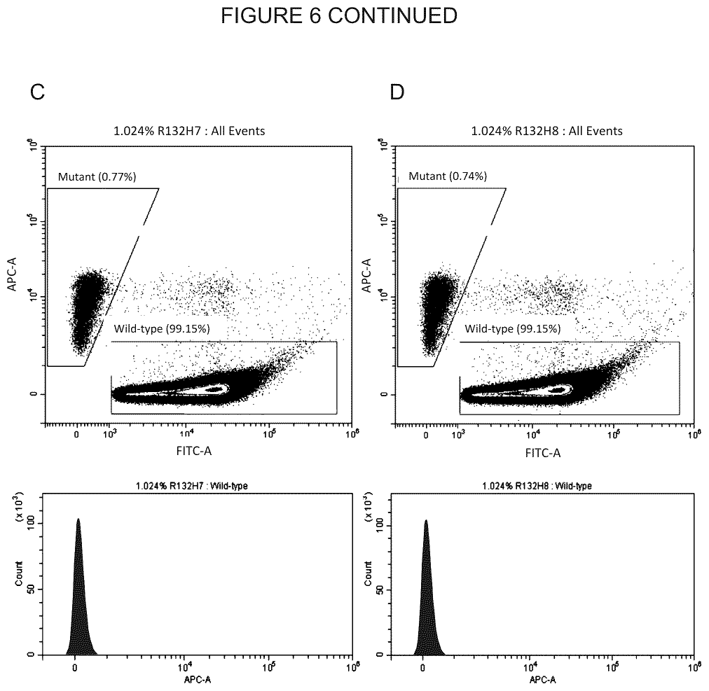

Various concentrations of BSA were used in single tube SuperRCA assays to analyse a sample with 1.024% of the R132H mutation. The SuperRCA products were detected using BD Fortessa flow cytometer (FIG. 6). FIG. 6 shows the flow cytometry results with 0.002% BSA, 0.005% BSA, 0.01% BSA or 0.02% BSA in the assay (by weight, or w/v). Tables 7 to 10 present the raw data from the flow cytometry readouts for each BSA concentration.

As can be seen from the data in FIG. 6 and Tables 7-10, presence of BSA in the SuperRCA assay at concentrations within the range of 0.002% to 0.02% (by weight, or w/v) helps to resolve the mutants and wild-type population by preventing them from clustering together in the solution; the absence of BSA would otherwise result in an increased population in the top-right corner of the graph (double positive population), an example of which can be seen in FIG. 2B. The presence of BSA, as shown in FIG. 6, helps to place more mutant events into the gate, increase the detected mutant frequency, and reducing the double positive events in the top-right corner. The number of events detected by flow cytometry for each reaction are displayed in Tables 7 to 10 below.

Tables 7 to 10 present the raw data from the flow cytometry readouts using, respectively, 0.002% BSA, 0.005% BSA, 0.01% BSA and 0.02% BSA (by weight, or w/v) as the resolving agent.

TABLE 7
(FIG. 6A)
R132H5 1.024% with 0.002% BSA
(by weight, or w/v)

| Population | Events | % Total |
|---|---|---|
| All Events | 1,045,487 | 100.00% |
| Mutant | 8,205 | 0.78% |
| Wild-type | 1,036,478 | 99.14% |

TABLE 8
(FIG. 6B)
R132H6 1.024% with 0.005% BSA
(by weight, or w/v)

| Population | Events | % Total |
|---|---|---|
| All Events | 987,703 | 100.00% |
| Mutant | 8,866 | 0.77% |
| Wild-type | 1,127,279 | 99.13% |

TABLE 9
(FIG. 6C)
R132H7 1.024% with 0.01% BSA
(by weight, or w/v)

| Population | Events | % Total |
|---|---|---|
| All Events | 1,137,224 | 100.00% |
| Mutant | 7,591 | 0.78% |
| Wild-type | 979,268 | 99.15% |

TABLE 10
(FIG. 6D)
R132H8 1.024% with 0.02% BSA
(by weight, or w/v)

| Population | Events | % Total |
|---|---|---|
| All Events | 988,963 | 100.00% |
| Mutant | 7,356 | 0.74% |
| Wild-type | 980,511 | 99.15% |

1. A method for detecting two or more differently labelled high molecular weight nucleic acid products in a mixture, wherein said products are detected by detecting one or more labels which are attached directly or indirectly to each product, said method comprising including in the mixture for detection a resolving agent to facilitate resolution of the differently labelled products, wherein said resolving agent acts to inhibit interaction between different labels.

2. A method of detecting two or more target nucleic acid sequences in one or more nucleic acid molecules in a sample, said method comprising:
(a) for each target nucleic acid sequence, generating a high molecular weight nucleic acid product as a detection assay reaction product for that sequence, and creating a reaction mixture comprising the resulting nucleic acid products for the two or more target nucleic acid sequences;
(b) providing each nucleic acid product of (a) with multiple copies of one or more labels, wherein each nucleic product is differently labelled and can be distinguished from another nucleic acid product, and wherein the labels are provided during the generation of the nucleic acid products in (a) or after, thereby generating a detection mixture comprising two or more differently labelled nucleic acid products, wherein a resolving agent is included in the detection mixture to facilitate resolution of the differently labelled products, wherein said resolving agent acts to inhibit interaction between different labels;
(c) detecting the labelled nucleic acid products in the detection reaction mixture, thereby detecting the target nucleic acid sequences.

3. The method of claim 1 or claim 2, wherein the resolving agent is or comprises a protein.

4. The method of any one of claims 1 to 3, wherein said protein is an inert protein.

5. The method of any one of claims 1 to 4, wherein the resolving agent is selected from non-fat dry milk or a milk protein, serum or a serum protein, gelatin, SSB or a peptide of random sequence.

6. The method of any one of claims 1 to 5, wherein the resolving agent is casein, a serum albumin, or a globulin, optionally an immunoglobulin.

7. The method of any one of claims 1 to 6, wherein the resolving agent is porcine serum albumin, bovine serum albumin, human serum albumin or bulk IgG.

8. The method of claim 1 or claim 2, wherein the resolving agent is not, or does not comprise, a protein.

9. The method of any one of claim 1, 2 or 8, wherein the resolving agent is a detergent or a non-amino acid polymer, optionally a polyvinyl alcohol.

10. The method of claim 9, wherein the resolving agent is selected from Polyvinylpyrrolidone (PVP) and Tween-20.

11. The method of any one of claims 1 to 10, wherein the high molecular weight nucleic acid product is a nucleic acid amplification product, a nucleic acid polymerisation product, or a nucleic acid hybridisation product.

12. The method of claim 11, wherein the nucleic acid product is a RCA product or a HCR product, or a product of a branched DNA amplification reaction.

13. The method of any one of claims 1 to 12, wherein the label is a spectrophotometrically-detectable label.

14. The method of claim 13, wherein the label is a fluorescent, coloured or colorimetric label.

15. The method of any one of claims 1 to 14, wherein the nucleic acid product is labelled by hybridising a labelled detection oligonucleotide thereto.

16. The method of any one of claims 1 to 15, wherein the resolving agent is included or present in one or more of the steps of generating the nucleic acid products.

17. The method of claim 16, wherein the nucleic acid product is a sRCA product and the resolving agent is included in the reaction mixture for the first RCA of the sRCA reaction in excess, and is retained in the reaction mixture for subsequent steps.

18. The method of any one of claims 15 to 17, wherein the resolving agent is included in a labelling mix comprising a mixture of labelled detection oligonucleotides.

19. The method of any one of claims 1 to 18, wherein the resolving agent is included in a labelling mix which is contacted with the mixture of nucleic acid products to label them, or is added to the mixture of nucleic acid products, prior to, simultaneously with or after the labelling mix, but prior to detecting the labelled nucleic acid products.

20. The method of any one of claims 1 to 19, wherein the nucleic acid product is a SuperRCA product generated using a target-specific padlock probe which hybridises to a first RCA product which comprises multiple repeats of a target nucleic sequence or a complementary copy thereof, wherein the padlock probes hybridised to the multiple repeats are circularised by ligation and subjected to RCA to generate a second RCA product.
